(19) United States
(12) Patent Application Publication
(10) Pub. No.: US 2005/0100538 A1
(43) Pub. Date: May 12, 2005

(54) Intradiscal injection of anti-oxidants

(76) Inventors: Attawia Mohamed, Canton, MA (US); DiMauro Thomas, Southboro, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/938,906

(22) Filed: Sep. 10, 2004

Related U.S. Application Data
(63) Continuation-in-part of application No. 10/631,487, filed on Jul. 31, 2003.

(51) Int. Cl. A61F 2/44; A61K 38/44; A61K 31/355; A61K 33/04; A61K 31/375; A61K 31/07

(52) U.S. Cl. 424/94.4; 623/17.16; 514/494; 424/702; 514/458; 514/474; 514/725

(57) ABSTRACT

A therapeutic method of delivering anti-oxidants to an intervertebral disc is disclosed.

### RELATED APPLICATIONS

This application is a continuation-in-part of U.S. patent application Ser. No. 10/631,487, filed Jul. 31, 2003, “Transdiscal Administration of Specific Inhibitors of p38 Kinase” (3518.1012-003).

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contained sulfated functional groups that retain water, thereby providing the nucleus pulposus within its cushioning qualities. These nucleus pulposus cells may also secrete small amounts of cytokines such as interleukin-1β and TNF-α as well as matrix metalloproteinases (MMPs). These cytokines and MMPs help regulate the metabolism of the nucleus pulposus cells.

In some instances of disc degeneration disease (DDD), gradual degeneration of the intervetebral disc is caused by mechanical instabilities in other portions of the spine. In these instances, increased loads and pressures on the nucleus pulposus cause the cells within the disc (or invading macrophages) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins that may cause nerve irritation and pain.

As DDD progresses, toxic levels of the cytokines and MMPs present in the nucleus pulposus begin to degrade the extracellular matrix (in particular, the MMPs (as mediated by the cytokines) begin cleaving the water-retaining portions of the proteoglycans, thereby reducing its water-retaining capabilities. This degradation leads to a less flexible nucleus pulposus, and so changes the loading pattern within the disc, thereby possibly causing delamination of the annulus fibrosus. These changes cause more mechanical instability, thereby causing the cells to emit even more cytokines, thereby upregulating MMPs. As this destructive cascade continues and DDD further progresses, the disc begins to bulge (“a herniated disc”), and then ultimately ruptures, causing the nucleus pulposus to contact the spinal cord and produce pain.

A significant portion of the invading macrophages are neutrophils. In addition to their emission of cytokines, the invading neutrophils also emit reactive oxygen species (ROS), such as hydroxyl radicals, superoxide ion and hydrogen peroxide. These ROS are believed to contribute the degradation of the matrix, thereby furthering the cleavage of the proteoglycans.

### SUMMARY OF THE INVENTION

It is believed that intradiscal administration of an effective amount of an anti-oxidant would also help provide therapy to the patient having DDD. It is believed that oxidants degrade the nucleus pulposus extra-cellular matrix. Typical anti-oxidants include free radical scavengers and superoxide dismutase enzymes.

Therefore, in accordance with another embodiment of the present invention, there is provided a method of treating degenerative disc disease in an intervertebral disc having a nucleus pulposus, comprising intradiscally administering an effective amount of a formulation comprising an antioxidant into an intervertebral disc.

In some embodiments, the anti-oxidant comprises Vitamin C. As a water-soluble antioxidant, vitamin C scavenges aqueous peroxyl radicals that participate in the lipid degradation process. It works along with vitamin E, a fat-soluble antioxidant, and glutathione peroxidase to stop free radical chain reactions. As an antioxidant, vitamin C's primary role is to neutralize free radicals. Since ascorbic acid is water soluble, it can work both inside and outside the cells to prevent free radical damage. Free radicals will seek out an electron to regain their stability. Vitamin C is an excellent source of electrons; therefore, it can donate electrons to free radicals such as hydroxyl and superoxide radicals and quench their reactivity. Vitamin C also works along with glutathione peroxidase to revitalize vitamin E, a fat-soluble antioxidant. In addition to its work as a direct scavenger of free radicals in fluids, then, vitamin C also contributes to the antioxidant activity in the lipids.

Tiku, J. Biol. Chem., 275(26), 20069-76, reports that Vitamin E or C, when administered in concentrations between 10 μM and 250 μM, significantly diminished the release of labeled matrix by activated cultured articular chondrocytes and concludes that Vitamin E or C has an anti-oxidant role in preventing protein oxidation. Kurz, Osteoarthritis and Cartilage, 202, 10, 119-126, provided a special diet to mice that included 1000 mg Vitamin C/kg and found a diet dependent increase in expression and activity of antioxidative molecules, as well as a parallel decrease in mechanical induction of osteoarthritis. Kurz concluded there there is a connection between vitamins and mechanically-induced OA.

In some embodiments, the anti-oxidant comprises Vitamin E. Vitamin E protects unsaturated fatty acids against oxidation. Vitamin E, a fat-soluble antioxidant, stops free radical chain reactions.

Tiku, J. Biol. Chem., 275(26), 20069-76, reports that Vitamin E, when administered in concentrations between 0.1 μM and 25 μM, somewhat diminished the release of labeled matrix by activated cultured articular chondrocytes.

Kurz, Osteoarthritis and Cartilage, 202, 10, 119-126, provided a special diet to mice that included 300 mg Vitamin E/kg and found a diet dependent increase in expression and activity of antioxidative molecules, as well as a parallel decrease in mechanical induction of osteoarthritis.

Kilic, Pediatric Hematol Oncol. 1998 July-August;15(4);339-46, reported intra-articularly injected 20 mg of Vitamin E into rabbits having hemarthrosis, found significantly decreased proteoglycan levels in this group, and concluded that Vitamin E may be helpful in preventing joint cartilage changes seen in hemarthrosis.

In some embodiments, the anti-oxidant is Vitamin A. Vitamin A is also known to be a powerful anti-oxidant.

In some embodiments, the anti-oxidant comprises a trace element, which is preferably copper, zinc or selenium. These trace elements act as anti-oxidants by virtue of their incorporation into specific anti-oxidant enzymes.

In some embodiments, the zinc-based anti-oxidant is catalase. According to Kamanli, Cell Biochem. Func. 2004, 22:53-57, catalase detoxifies hydrogen peroxide and converts lipid hydroperoxides into non-toxic alcohols, and is essential for the inhibition of inflammation related to the function of neutrophils.

It has been reported by Schalkwijk, J. Clin. Invest. 76, July 1985, 198-205, that intra-articular injection of catalase into the arthritic knee of a mouse suppressed some parameters of the inflammatory response. Salin, J. Clin. Invest., 56, November 1975, 1319-1323, investigated the extent to which CAT protected cells from superoxide ion and found that 250 μg CAT/ml was effective in protecting leukocytes. Tiku, Free Rad. Res., 30, 395-405, 1999, reports that about 300 U/ml (100-1000 g/ml) of catalase inhibits aggrecan degradation by LPS-stimulated chondrocytes and concludes that anti-oxidants can prevent matrix degradation.

In some embodiments, the selenium-based anti-oxidant is GSH-PX. According to Kamanli, supra, catalase detoxifies hydrogen peroxide and converts lipid hydroperoxides into non-toxic alcohols, and is essential for the inhibition of inflammation related to the function of neutrophils.

Kurz, Osteoarthritis and Cartilage, 202, 10, 119-126, provided a special diet to mice that included 2 mg Na₂SeO₃/kg and found a diet dependent increase in expression and activity of antioxidative molecules, as well as a parallel decrease in mechanical induction of osteoarthritis.

In some embodiments, the copper-based anti-oxidant is superoxide dismutase (SOD). According to Kamanli, supra, SOD catalyses dismutation of the superoxide anion into hydrogen peroxide.

Salin, J. Clin. Invest., 56, November 1975, 1319-1323, investigated the extent to which SOD protected cells from superoxide ion and found that 250 μg SOD/ml was effective in protecting leukocytes.

In some embodiments, the anti-oxidant comprises an iron-binding agent, which is preferably transferring or lactoferrin. These iron binding agents act as anti-oxidants by virtue of their ability to bind free iron. Since iron is an important catalyst in the conversion of hydrogen peroxide and superoxide ions into the more potent hydroxyl radical, iron-binding agents prevent the generation of more potent oxidative species.

Guillen, Arthritis. Rheum., 43, 2000, 2073-80 reports intra-articularly injecting 0.5-1 mg of lactoferrin into the knees of mice, reports significant suppression of local inflammation for up to 3 days, and concludes that lactoferrin is a potentially useful anti-inflammatory agent.

Trif, Exp. Biol. Med (Maywood), Exp. Biol. Med. 226(6):559-64, 2001 reports intra-articularly injecting 20 μg/ml of lactoferrin into the knees of mice for the purpose of preventing arthritis induced inflammation.

Hayashida, J. Vet. Med. Sci., 66(2), 149-154, 2004 (Hayashida I) reports that injecting 30-100 mg/kg lactoferrin into adjuvant arthritis rats and finding that the lactoferrin injection suppressed both TNF-a levels and the development of arthritis, while increasing IL-10 levels.

Importantly, Hayashida I also reported that the lactoferrin injection produced a very significant and dose-dependent analgesia. Therefore, it appears that iron-binding agents are especially attractive for use in DDD because they not only stop inflammation but they also may alleviate pain.

Hayashida, Eur. J. Pharmacology, 484, 2004, 175-181, reported that lactoferrin exerts an anti-nociceptive activity via potentiation of the peripheral μ-opiodergic system.

Biemond, Arthr. Rheum., 27(7) July 1984, 760-765 reports that ceruloplasmin accounts for about 70% of the protective capacity of serum or synovial fluid of RA patients.

### DETAILED DESCRIPTION

Vitamins C, A and E may be readily obtained from numerous sources. For example, in some embodiments, Vitamin E is is obtainable from Sigma Chemical (St. Louis, Mo.).

In some embodiments, the trace element-based enzyme is made cationic, preferably by either coupling with polylysine or shielding anionic sites. Schalkwijk, J. Clin. Invest. 76, July 1985, 198-205, reported that injection of cationic catalase or peroxidase induced a marked suppression of some parameters of the inflammatory response.

In some embodiments, the trace element-based anti-oxidant is provided exogenously. Preferably, the exogenous trace element based anti-oxidant is a recombinant anti-oxidant. More preferably, the exogenous catalase is obtainable from Sigma Chemical (St. Louis, Mo.); exogenus SOD is obtainable from Sigma Chemical (St. Louis, Mo.).

In some embodiments, the trace-element based enzyme is derived autologously (i.e., from the patient). Preferably, the trace-element based enzyme is derived from the red blood cells of the patient. In some embodiments, autologous red blood cell lysate is used as the formulation comprising an effective amount of a trace element-based anti-oxidant. In others, the red blood cell lysate (obtained by centrifuging the patient's blood) undergoes at least partial purification prior to its intradiscal administration.

Conventional trace-element based enzyme purification technology further includes a number of unit processes designed to partially purify the concentration of trace element based enzyme. Such conventional processes include the use of glass beads to capture the trace-element based enzyme; the use of a 10 kD filter to capture the trace-element based enzyme; the use of a molecular sieve to dewater the crude lysate; the use of ammonium sulfate to precipitate out the trace-element based enzyme (Awasthi, JBC, 250(13), 5144; and Yang, JBC, 262(27) 13372); the use of column chromatography using phenyl-sepharose (Abbas, ABC, 2003, 377, 1026; Maddipati, JBC, 262, 36, 17398); or DEAE (Awasthi, supra; and Martinez, Thromb. Res. 19, 1980, 73-83) to separate out the enzyme; and the use of ethanol extraction to precipitate out the trace-element based enzyme (U.S. Pat. No. 4,341,867—Johansen).

In one preferred embodiment, co-isolation of GSH-PX, SOD and CAT is provided by the methods disclosed in Stepnik, J. Biochem. Biophys. Methods, 20, 1990, 157-169, the specification of which is incorporated by reference in its entirety.

For example, the purification processes disclosed in U.S. Pat. No. 4,341,867 (Johansen) and U.S. Pat. No. 4,435,506 (Jackson), the specification of which is hereby incorporated by reference in their entireties, may be selected as the respective catalase and SOD purification processes. It is reasonable to expect that adoption of at least one of the partial purification techniques described above will lead to a 5-10 fold increase in the trace-element based enzyme concentration in the partially purified solution. For example, Awasthi reports that ammonium sulfate precipitation yields 17- and 61-fold increases in enzyme concentration, while Martinez reports that column chromatography yields an 84-fold increase in enzyme concentration using DEAE Sepharose.

In some embodiments, the autologously derived trace element based enzyme is purified by captured by and elution from an antibody, preferably a monoclonal antibody.

In some embodiments, the iron-binding agent is provided exogenously. Preferably, the exogenous iron-binding agent is a recombinant iron-binding agent. More preferably, human apo-transferrin (20 mg/ml) is obtainable from Sigma, (Poole, UK); and the exogenous iron-free lactoferrin is obtainable from Sigma Chemical (St. Louis, Mo.); and the recombinant lactoferrin is obtainable from Tatua (Morrinsville, NZ).

In some embodiments, the iron-binding agent is derived autologously (i.e., from the patient). When the iron binding agent is transferrin, the iron-binding agent is preferably derived from the serum or plasma of the patient. In some embodiments thereof, autologous serum is used as the formulation comprising an effective amount of tranferrin (as it contains about 3 mg/ml of transferrin). In others, the autologous serum undergoes at least partial purification to concentration the transferring prior to its intradiscal administration. When the iron binding agent is lactoferrin, it is preferably derived from white blood cells present in the buffy coat of the patient's blood.

In some embodiments, the autologously derived iron-binding agent is purified by captured by and elution from an antibody, preferably a monoclonal antibody. For example, in one preferred embodiment, transferrin and its antibody (CD71) are allowed to complex, and the complex is captured by immobilized IgG, as in Desai, Anal. Biochem., 2004, May 15, 328(2) 162-5.

When injecting volumes into the nucleus pulposus, it is desirable that the volume of drug delivered be no more than 1 ml, preferably no more than 0.5 ml, more preferably between 0.1 and 0.3 ml. When injected in these smaller quantities, it is believed the added volume will not cause an appreciable pressure increase in the nucleus pulposus. Since an effective intradiscal administration of the anti-oxidant desirably arrests oxidation of the nucleus pulposus, and the typical lumbar nucleus pulposus has a volume of about 3 cc, it is believed that the intradiscal injection will occupy about 10% of the nucleus pulposus. Accordingly, the concentration of the bolus of anti-oxidant delivered to the nucleus pulposus should be at least about 10 times the concentration at which anti-inflammatory activity is expected to take place.

It is believed that as little as 10 μM Vitamin C is an effective anti-inflammatory concentration. Accordingly, the formulation comprising an effective amount of Vitamin C comprises at least 100 μM, more preferably at least 250 μM, more preferably at least 500 μM Vitamin C.

It is believed that as little as 5 μM Vitamin E is an effective anti-inflammatory concentration. Accordingly, the formulation comprising an effective amount of Vitamin E comprises at least 50 μM, more preferably at least 100 μM, more preferably at least 200 μM Vitamin E.

It is believed that as little as 100 U catalase/ml is an effective anti-inflammatory concentration. Accordingly, the formulation comprising an effective amount of catalase comprises at least 1000 U Catalase/ml, more preferably at least 3000 U Catalase/ml, more preferably at least 5000 U Catalase/ml.

It is believed that as little as 2 μg/ml GSH-PX is an effective anti-inflammatory concentration. Accordingly, the formulation comprising an effective amount of GSH-Px comprises at least 20 μg/ml, more preferably at least 50 μg/ml, more preferably at least 50 μg/ml.

It is believed that as little as 100 μg/ml SOD is an effective anti-inflammatory concentration. Accordingly, the composition comprising an effective amount of SOD comprises at least 1000 μg SOD/ml, more preferably at least 2500 μg/ml, more preferably at least 5000 μg/ml.

It is believed that as little as 20 p/ml of transferrin or lactoferrin is an effective anti-inflammatory concentration. Accordingly, the composition comprising an effective amount of transferrin or lactoferrin comprises at least 200 μg/ml, more preferably at least 500 μg/ml, more preferably at least 1000 μg/ml.

In some embodiments, adjunct materials disclosed in U.S. patent application Ser. No. 10/631,487, filed Jul. 31, 2003, “Transdiscal Administration of Specific Inhibitors of p38 Kinase”, the specification of which is incorporated by reference in its entirety, are provided along with the anti-oxidant.

Therefore, in accordance with the present invention, there is provided a method of treating degenerative disc disease in an intervertebral disc having a nucleus pulposus, comprising:
a) administering an anti-oxidant into a degenerating disc; and
b) administering at least one additional therapeutic agent in an amount effective to at least partially repair the disc.

In some embodiments, the additional agent is fibrin, hyaluronic acid, stem cells, bone marrow, platelet rich plasma, or a growth factor (in particular, rh GDF-5).

It is further believed that the forumulations of the present invention could be more effective in treating DDD when it includes a second anti-oxidant. In particular, it is observed by the present inventors that various anti-oxidants do not act upon ROS via the same mechanism, but rather act upon ROS by different mechanisms. Therefore, the inclusion of a second anti-oxidant may increase the effectiveness of the formulation.

In some embodiments, the first anti-oxidant comprises a first vitamin and the second anti-oxidant comprises a second vitamin. In some embodiments, the first anti-oxidant comprises a Vitamin C and the second anti-oxidant comprises Vitamin E. In some embodiments, the first anti-oxidant comprises a vitamin and the second anti-oxidant comprises a trace element. In some embodiments, the vitamin comprises Vitamin C and the trace element comprises zinc. In some embodiments, the vitamin comprises Vitamin C and the trace element comprises copper. In some embodiments, the vitamin comprises Vitamin C and the trace element comprises selenium. In some embodiments, the vitamin comprises Vitamin E and the trace element comprises zinc. In some embodiments, the vitamin comprises Vitamin E and the trace element comprises copper. In some embodiments, the vitamin comprises Vitamin E and the trace element comprises selenium. In some embodiments, the first anti-oxidant comprises a first trace element and the second anti-oxidant comprises a second trace element. In some embodiments, the first anti-oxidant comprises a copper and the second anti-oxidant comprises zinc. In some embodiments, the first anti-oxidant comprises copper and the second anti-oxidant comprises selenium. In some embodiments, the first anti-oxidant comprises zinc and the second anti-oxidant comprises selenium.

In addition, it has been further observed that the different anti-oxidants often act upon ROS in concert upon different components of the ROS.

For example, although catalase detoxifies superoxide anion, the resulting product of the detoxification (hydrogen peroxide) may still degrade the ECM. Since each of GSH-PX and catalase convert hydrogen peroxide, it is believed that there is special advantage in a formulation include a) SOD, and b) at least one of GSH-PX and catalase.

Modifications of the anti-oxidant and its functional fragments that either enhance or do not greatly affect the ability to inhibit oxidation are also included within the term “anti-oxidant.” Such modifications include, for example, additions, deletions or replacements of one or more amino acids from the native amino acid sequence of an enzyme anti-oxidant or iron-binding agent with a structurally or chemically similar amino acid or amino acid analog. These modifications will either enhance or not significantly alter the structure, conformation or functional activity of the anti-oxidant or a functional fragment thereof. Modifications that do not greatly affect the activity of the anti-oxidant or its functional fragments can also include the addition or removal of sugar, phosphate or lipid groups as well as other chemical derivations known in the art. Additionally, anti-oxidant or its functional fragments can be modified by the addition of epitope tags or other sequences that aid in its purification and which do not greatly affect its activity. As used herein, the term “functional fragment,” in connection with an anti-oxidant, is intended to mean a portion of the anti-oxidant that maintains the ability of the anti-oxidant to inhibit oxidiation. A functional fragment can be, for example, from about 6 to about 300 amino acids in length, for example, from about 7 to about 150 amino acids in length, more preferably from about 8 to about 50 amino acids in length. If desired, a functional fragment can include regions of the anti-oxidant with activities that beneficially cooperate with the ability to inhibit oxidation. For example, a functional fragment of the anti-oxidant can include sequences that promote the ingrowth of cells, such as endothelial cells and macrophages, at the site of inflammation.

Vitamin C is defined to include ascorbic acid and its derivatives. Vitamin E is defined to include alpha-tocopherol and its derivatives. Vitamin A is defined to include all-trans-retinoic acid and its derivatives.

In some embodiments, the formulation of the present invention may be housed in the barrel of a syringe and delivered by injection through a needle into the interveterbal disc of a patient.

Preferably, the formulation of the present invention is injected into the disc through a small bore needle. More preferably, the needle has a bore of about 22 gauge or less, so that the possibilities of producing a herniation are mitigated. For example, the needle can have a bore of about 24 gauge or less, so that the possibilities of producing a herniation are even further mitigated.

In preferred embodiments, the formulation of the present invention is administered directly into the disc through the outer wall of the annulus fibrosus. In one embodiment, the direct administration includes depositing the anti-oxidant in the nucleus pulposus portion of the disc. In this condition, the fibrous nature of the annulus fibrosus that surrounds the nucleus pulposus will help keep the anti-oxidant contained within the disc.

In some embodiments, the formulation of the present invention may be delivered by iontophoresis. Iontophoresis uses an electrical voltage as a driving force to move ionized species. Since the anti-oxidants comprising trace elements generally have a positive charge in water, it is believed that iontophoresis may be used to administer these anti-oxidants to the disc without invasion of the disc.

In some embodiments, the formulation of the present invention may be delivered by electroporation. Electroporation provides a short term, pulsed electrical voltage across a tissue to temporarily breakdown cell membranes within the tissue, thereby enhancing the permeability of those cells for drug delivery purposes. Accordingly, electroporation may be used to deliver anti-oxidants into the disc without invasion of the disc.

### EXAMPLE I

This non-limiting prophetic example describes how to intradiscally administer a formulation comprising an anti-oxidant into a nucleus pulposus of a degenerating disc.

First, a clinician uses a diagnostic test to verify that a particular disc within a patient has high levels of a particular ROS.

Next, the clinician provides a local anesthetic (such as 5 ml lidocaine) to the region dorsal of the disc of concern to reduce subcutaneous pain.

Next, the clinician punctures the skin of the patient dorsal the disc of concern with a relatively large (e.g., 18-19 gauge) needle having a stylet therein, and advances the needle through subcutaneous fat and dorsal sacrolumbar ligament and muscles to the outer edge of the intervertebral disc.

Next, the stylet is removed from the needle.

Next, the clinician receives a syringe having a smaller gauge needle adapted to fit within the larger gauge needle. This needle is typically a 22 or 24 gauge needle. The barrel of the syringe contains the formulation of the present invention.

The formulation contains lactoferrin as an anti-oxidant, and has a concentration of between about 1 mg/ml and about 10 mg/ml.

Next, the physician advances the smaller needle co-axially through the larger needle and past the distal end of the larger needle, thereby puncturing the annulus fibrosus. The smaller needle is then further advanced into the center of the nucleus pulposus. Finally, the clincian depresses the plunger of the syringe, thereby injecting between about 0.1 and 1 ml of the formulation into the nucleus pulposus.

1. A method of treating degenerative disc disease in an intervertebral disc having a nucleus pulposus, comprising transdiscally administering an effective amount of a formulation comprising a first antioxidant into an intervertebral disc.

2. The method of claim 1 wherein the anti-oxidant is a vitamin.

3. The method of claim 2 wherein the vitamin is Vitamin C.

4. The method of claim 3 wherein the formulation comprises at least 100 μM Vitamin C.

5. The method of claim 2 wherein the vitamin is Vitamin E.

6. The method of claim 5 wherein the vitamin is Vitamin A.

7. The method of claim 1 wherein the anti-oxidant comprises a trace element.

8. The method of claim 7 wherein the trace element is selected from the group consisting of copper, zinc and selenium.

9. The method of claim 8 wherein the trace element is copper.

10. The method of claim 9 wherein the copper is incorporated in superoxide dismutase (SOD).

11. The method of claim 10 wherein the formulation comprises at least 1000 U/ml SOD.

12. The method of claim 8 wherein the trace element is selenium.

13. The method of claim 12 wherein the selenium is incorporated in GSH-PX.

14. The method of claim 13 wherein the formulation comprises at least 20 μg/ml GSH-PX.

15. The method of claim 8 wherein the trace element is zinc.

16. The method of claim 12 wherein the zinc is incorporated in catalase.

17. The method of claim 13 wherein the formulation comprises at least 1000 U/ml catalase.

18. The method of claim 1 wherein the formulation comprises a second anti-oxidant.

19. The method of claim 18 wherein the first anti-oxidant comprises a first vitamin and the second anti-oxidant comprises a second vitamin.

20. The method of claim 18 wherein the first anti-oxidant comprises a Vitamin C and the second anti-oxidant comprises Vitamin E.

21. The method of claim 18 wherein the first anti-oxidant comprises a vitamin and the second anti-oxidant comprises a trace element.

22. The method of claim 21 wherein the vitamin comprises Vitamin C and the trace element comprises zinc.

23. The method of claim 21 wherein the vitamin comprises Vitamin C and the trace element comprises copper.

24. The method of claim 21 wherein the vitamin comprises Vitamin C and the trace element comprises selenium.

25. The method of claim 21 wherein the vitamin comprises Vitamin E and the trace element comprises zinc.

26. The method of claim 21 wherein the vitamin comprises Vitamin E and the trace element comprises copper.

27. The method of claim 21 wherein the vitamin comprises Vitamin E and the trace element comprises selenium.

28. The method of claim 18 wherein the first anti-oxidant comprises a first trace element and the second anti-oxidant comprises a second trace element.

29. The method of claim 28 wherein the first anti-oxidant comprises a copper and the second anti-oxidant comprises zinc.

30. The method of claim 28 wherein the first anti-oxidant comprises copper and the second anti-oxidant comprises selenium.

31. The method of claim 28 wherein the first anti-oxidant comprises zinc and the second anti-oxidant comprises selenium.

32. A syringe having a barrel containing a formulation comprising a first antioxidant and a second antioxidant.

33. The syringe of claim 32 wherein the first anti-oxidant comprises a first vitamin and the second anti-oxidant comprises a second vitamin.

34. The syringe of claim 32 wherein the first anti-oxidant comprises a Vitamin C and the second anti-oxidant comprises Vitamin E.

35. The syringe of claim 32 wherein the first anti-oxidant comprises a vitamin and the second anti-oxidant comprises a trace element.

36. The syringe of claim 35 wherein the vitamin comprises Vitamin C and the trace element comprises zinc.

37. The syringe of claim 35 wherein the vitamin comprises Vitamin C and the trace element comprises copper.

38. The syringe of claim 35 wherein the vitamin comprises Vitamin C and the trace element comprises selenium.

39. The syringe of claim 35 wherein the vitamin comprises Vitamin E and the trace element comprises zinc.

40. The syringe of claim 35 wherein the vitamin comprises Vitamin E and the trace element comprises copper.

41. The syringe of claim 35 wherein the vitamin comprises Vitamin E and the trace element comprises selenium.

42. The syringe of claim 32 wherein the first anti-oxidant comprises a first trace element and the second anti-oxidant comprises a second trace element.

43. The syringe of claim 42 wherein the first anti-oxidant comprises a copper and the second anti-oxidant comprises zinc.

44. The syringe of claim 42 wherein the first anti-oxidant comprises copper and the second anti-oxidant comprises selenium.

45. The syringe of claim 42 wherein the first anti-oxidant comprises zinc and the second anti-oxidant comprises selenium.

46. A syringe having a barrel containing a formulation comprising a first antioxidant comprising a trace element.

47. The syringe of claim 46 wherein the trace element is selected from the group consisting of zinc, copper and selenium.

48. The syringe of claim 47 wherein the trace element is zinc.

49. The syringe of claim 48 wherein the first anti-oxidant is SOD.

50. The syringe of claim 47 wherein the trace element is copper.

51. The syringe of claim 48 wherein the first anti-oxidant is catalase.

52. The syringe of claim 47 wherein the trace element is selenium.

53. The syringe of claim 48 wherein the first anti-oxidant is GSH-PX.

54. The syringe of claim 46 wherein the formulation further comprises a second antioxidant comprising a second trace element.

55. A syringe having a barrel containing a formulation comprising an antioxidant comprising an iron binding agent.

56. The syringe of claim 55 wherein the iron binding agent is selected from the group consisting of transferring and lactoferrin.

57. The syringe of claim 56 wherein the iron binding agent is transferrin.

58. The syringe of claim 48 wherein the transferrin is autologous.

59. The syringe of claim 47 wherein the iron binding agent is lactoferrin.

60. The syringe of claim 48 wherein the lactoferrin is autologous.

61. A method of treating degenerative disc disease in an intervertebral disc of a patient having a nucleus pulposus and an annulus fibrosus, comprising the steps of:
a) intradiscally administering an effective amount of a formulation comprising an iron-binding agent into the intervertebral disc.

62. The method of claim 61 wherein the agent is autologous.

63. The method claim 61 wherein the agent is obtained from the patient's blood.

64. The method of claim 61 wherein the agent is exogenous.

65. The method of claim 61 wherein the agent is transferrin.

66. The method of claim 65 wherein the transferrin is autologous.

67. The method of claim 65 wherein the transferrin is exogenous.

68. The method of claim 61 wherein the agent is lactoferrin.

69. The method of claim 68 wherein the lactoferrin is autologous.

70. The method of claim 68 wherein the lactoferrin is exogenous.

71. The method of claim 61 wherein the iron-binding agent is an anti-oxidant

72. The method of claim 61 wherein the iron binding agent is administered in an amount effective to alleviate pain.

73. The method of claim 61 wherein the iron binding agent is administered in a concentration of at least about 3 mg/ml.

74. The method of claim 61 wherein the iron binding agent is administered in an amount of at least about 1 mg.

75. The method of claim 61 wherein the formulation comprises fibrin.

76. The method of claim 61 wherein the formulation comprises hyaluronic acid

77. The method of claim 61 wherein the formulation comprises stem cells.

78. The method of claim 61 wherein the formulation comprises platelet rich plasma.

79. The method of claim 61 wherein the formulation comprises a growth factor.

80. The method of claim 61 wherein the formulation comprises rh-GDF-5.

81. A method of treating degenerative disc disease in an intervertebral disc having a nucleus pulposus, comprising:
a) administering an effective amount of an anti-oxidant into a degenerating disc; and
b) administering at least one additional therapeutic agent in an amount effective to at least partially repair the disc.
